(19) United States
(12) Patent Application Publication
(10) Pub. No.: US 2008/0208081 A1
(43) Pub. Date: Aug. 28, 2008

(54) System and Method For Determining Tibial Rotation

(75) Inventors: Stephen B. Murphy, Winchester, MA (US); Daniel L. McCombs, Paw Paw, MI (US)

Correspondence Address: CHIEF PATENT COUNSEL;SMITH & NEPHEW, INC., 1450 BROOKS ROAD, MEMPHIS, TN, 38116, US

(73) Assignee: SMITH & NEPHEW, INC., Memphis, TN (US)

(21) Appl. No.: 11/913,447

(22) PCT Filed: May 2, 2006
(86) PCT No.: PCT/US06/17042

§ 371 Date: Nov. 2, 2007

Related U.S. Application Data
(60) Provisional application No. 60/677,399, filed on May 2, 2005.

(51) Int. Cl. A61B 5/103; G01N 33/48

(52) U.S. Cl. 600/595; 702/19

(57) ABSTRACT

A system and method for determining tibial rotation is disclosed. The system includes a first fiducial, a second fiducial, a position and orientation sensor, a computer, and a monitor. The first fiducial is connected to a first part, and the second fiducial is connected to a second part. The position and orientation sensor tracks the first fiducial and the second fiducial. The computer has a memory, a processor, and an input/output device. The input/output device receives data from the position and orientation sensor. The processor processes the data to identify a first axis of the first part and a second axis of the second part. The processor constructs a reference plane through the second axis and orthogonal to the first axis. The monitor is connected to the input/output device and displays a rendering of the reference plane.

### CROSS-REFERENCE TO RELATED APPLICATIONS

This application claims the benefit of U.S. Provisional Application No. 60/677,399, filed 2 May 2005.

### STATEMENT REGARDING FEDERALLY SPONSORED RESEARCH OR DEVELOPMENT

Not Applicable.

### APPENDIX

Not Applicable.

### BACKGROUND OF THE INVENTION

1. Field of the Invention

This invention relates generally to computer assisted surgery and more particularly to a system for computer assisted surgery utilizing a projected method for determining tibial rotation.

2. Related Art

During knee arthroplasty, one or more of the distal surfaces of the femur are cut away and replaced with a metal component to simulate the bearing surfaces of the femur. Similarly, one or more of the proximal surfaces of the tibia is modified to provide a metal-backed plastic bearing surface. The metal femoral component of the new prosthetic joint transfers the weight of the patient to the tibial component such that the joint can support the patient's weight and provide a near-normal motion of the knee joint.

Orthopedic surgeons have been struggling with the alignment of knee arthroplasties since their inception in the early 1970s. Basically, what is generally necessary is a 5-7 degree angular resection of the distal femoral condyles as related to the mechanical axis of the femur and a perpendicular resection of the proximal tibia as related to its central axis. Early on, resections of the distal femur and proximal tibia were made by visually trying to match or correct the existing anatomy by eye. Alignment varied considerably depending on the skill of the operating surgeon.

Several studies have indicated that the long term performance of a prosthetic knee joint is dependant on how accurately the components of the knee joint are implanted with respect to the weight bearing axis of the patient's leg. The most important parameter in achieving long term performance is accurate alignment of the components. It has been proven that only 4.5 degrees of misalignment causes the components to only load one side of the knee joint leading to rapid failure of the implant. The literature strongly supports the conclusion that the closer the surgeons approach neutral alignment, the more successful the implant system will be with longevity. Misaligned knee arthroplasties tend to get worse with time because the abnormal weight distribution accelerates the wear on the overloaded side leading to rapid failure within a few years in the case of the gross malalignment.

In a correctly functioning knee, the weight bearing axis passes through the center of the head of the femur, the center of the knee and the center of the ankle joint. This weight bearing axis typically is located by analyzing an X-ray image of the patient's leg, taken while the patient is standing. The X-ray image is used to locate the center of the head of the femur and to calculate the position of the head relative to selected landmarks on the femur. The selected landmarks are then found on the patient's femur during surgery and the calculations used to estimate the actual position of the femoral head. These two pieces of information are used to determine the correct alignment of the weight bearing axis for the femur, commonly referred to as the mechanical axis of the femur. To completely define the correct position for the femoral component of the knee prosthesis, the correct relationship between the center of the femoral head and the knee joint and the rotation of the knee joint about the mechanical axis must be established. This information is determined from landmarks on the distal portion of the femur. The correct alignment for the tibial component of the knee prosthesis ordinarily is determined by finding the center of the ankle joint and relating its position to landmarks on the tibia. This point and the center of the proximal tibial plateau are used to define the weight bearing axis, or mechanical axis, of the tibia. The correct relationship between the ankle joint and the knee joint and the rotation of the knee joint about the mechanical axis are determined by reference to the distal portion of the femur and landmarks on the tibial plateau.

Presently, doctors commonly determine a desired rotation of the tibia simply by placing the knee in full extension and looking at the alignment of the foot. This method has several deficiencies. First, any errors that are developed in the determination of the femur's rotational axis are projected onto the tibia. Second, this method is much more susceptible to anatomic abnormalities and joint instability, which is common in patients requiring total knee arthroplasty. Third, a good rotational assessment of the tibia itself is not accurately determined, but rather, the entire rotation of the limb is being assessed in aggregate, without specific knowledge of the rotation of the tibia itself or the tibial component.

Other methods currently used to determine the Anterior-Posterior (AP) axis of the tibia rely on anatomic landmarks. One common method uses a line drawn from the medial ⅓ of the tibial tubercle to the center of the tibial plateau. Another method uses a line drawn from the anterior cruciate ligament insertion to the posterior cruciate ligament insertion. Still another method considers the average of these two or lines drawn from other landmarks, which assumes that averaging of these methods adds credence to the result. Ultimately though, because these points are all very close to each other in space, these methods are greatly affected by very small changes in their perceived location and thus are poorly reproduceable.

In yet another method, the rotation of both the femur and tibia is determined by developing a kinematic axis in the knee joint. This method requires the limbs to be moved with respect to each other, during which software determines the axis about which the tibia rotates with respect to the femur. Software then uses this axis for measuring rotation around the mechanical axis of the tibia and femur. The problem with this method is that it is extremely sensitive to anatomic abnormalities, as well as ligament instability.

For some time, computer assisted surgery (also known as “image-guided surgery,” “surgical navigation,” or “3-D computer surgery”) has been applied to invasive surgical procedures, such as knee arthroplasty. Computer assisted surgery, often abbreviated CAS, typically includes systems and processes for tracking anatomy, implements, instrumentation, trial implants, implant components and virtual constructs or references, and rendering images and data related to them in connection with orthopedic, surgical and other operations. CAS allows for the association of anatomical structures, constructs, and points-in-space with a fiducial. Fiducial functionality allows the CAS system to sense and track the position and orientation of these items. Such structures, items and constructs can be rendered onscreen properly positioned and oriented relative to each other using associated image files, data files, image input, and other sensory input based on the tracking. The CAS system, among other things, allow surgeons to navigate and perform knee arthroplasty using images that reveal interior portions of the body combined with computer generated or transmitted images that show surgical implements, instruments, trials, implants, and/or other devices located and oriented properly relative to the body part. By using the CAS system, the surgeon can accurately and effectively resection bones, place and assess trial implants and joint performance, and place and assess actual implants and joint performance.

There remains a need in the art for computer assisted surgery system that enables surgeons to accurately and reliably perform knee arthroplasty. In particular, there remains a need in the art for a computer assisted surgery system that allows a user to identify an angular rotation of an item, such as a tool, relative to the mechanical axis of a tibia.

### SUMMARY OF THE INVENTION

It is in view of the above problems that the present invention was developed. The invention is a system and method for determining tibial rotation. The invention has several advantages over prior devices and techniques. First, the invention has improved accuracy over the art. The invention utilizes the mechanical axis of the femur and the mechanical axis of the tibia to construct a reference plane. Because the endpoints of each axis are not in proximity to each other, small errors in their respective identification do not greatly affect the determination of the reference plane. Moreover, anatomic defects are less likely to effect the rotational position of the tibia. Second, the simplicity of the invention allows it to be easily repeatable. Surgeons are intimately familiar with finding the mechanical axis of the femur and the tibia and significant effort is not required to put the axes in 90 degrees of flexion. The simple and straightforward character of the invention allows it to be carried out by both new and experienced users.

Thus, in furtherance of the above goals and advantages, the present invention is, briefly, a system for performing computer assisted surgery. The system comprises: a first fiducial operatively connected to a first part; a second fiducial operatively connected to a second part; at least one position and orientation sensor adapted to track said first fiducial and said second fiducial; a computer having a memory, a processor, and an input/output device, said input/output device adapted to receive data from said at least one position and orientation sensor relating to a position and an orientation of said first fiducial and said second fiducial, said processor adapted to process said data to identify a first axis of the first part and a second axis of the second part, and said processor adapted to construct a reference plane through said second axis and orthogonal to said first axis; and a monitor operatively connected to said input/output device of said computer, and wherein said monitor is adapted to display a rendering of said reference plane.

Further features, aspects, and advantages of the present invention, as well as the structure and operation of various embodiments of the present invention, are described in detail below with reference to the accompanying drawings.

### BRIEF DESCRIPTION OF THE DRAWINGS

The accompanying drawings, which are incorporated in and form a part of the specification, illustrate the embodiments of the present invention and together with the description, serve to explain the principles of the invention. In the drawings:

FIG. 1 is a schematic view of a computer assisted surgery system;

FIG. 2 is a view of a knee prepared for surgery, including a femur and a tibia, to which fiducials have been attached;

FIG. 3 is a view of a portion of a leg prepared for surgery with a C-arm for obtaining fluoroscopic images associated with a fiducial;

FIG. 4 is a fluoroscopic image of free space rendered on a monitor;

FIG. 5 is a fluoroscopic image of femoral head obtained and rendered;

FIG. 6 is a fluoroscopic image of a knee obtained and rendered;

FIG. 7 shows a probe being used to register a surgically related component for tracking;

FIG. 8 shows a probe being used to register a cutting block for tracking;

FIG. 9 shows a probe being used to register a tibial cutting block for tracking;

FIG. 10 shows a probe being used to register a femoral cutting block for tracking;

FIG. 11 shows a probe being used to designate landmarks on bone structure for tracking;

FIG. 12 is another view of a probe being used to designate landmarks on bone structure for tracking;

FIG. 13 is another view of a probe being used to designate landmarks on bone structure for tracking;

FIG. 14 is a screen face produced during designation of landmarks to determine a femoral mechanical axis;

FIG. 15 is a screen face produced during designation of landmarks to determine an epicondylar axis;

FIG. 16 is a screen face produced during designation of landmarks to determine an anterior-posterior axis;

FIG. 17 is a screen face that presents graphic indicia which may be employed to help determine reference locations within bone structure;

FIG. 18 is a screen face showing mechanical and other established axes;

FIG. 19 is a schematic view of a patient's leg;

FIG. 20 is an illustration of a screen face displaying degrees of flexion;

FIG. 21 is a flowchart illustrating software steps for tracking and using a tibial rotation plane;

FIG. 22 is a schematic front view of a patient's leg;

FIG. 23 is a schematic medial side view of a patient's leg;

FIG. 24 is a schematic front view of a femur;

FIG. 25 is a schematic medial side view of a femur;

FIG. 26 is a schematic front view of a patient's leg;

FIG. 27 is a schematic medial side view of a patient's leg;

FIG. 28 is another screen face showing mechanical and other established axes;

FIG. 29 is another screen face showing mechanical and other established axes;

FIG. 30 shows navigation and placement of an intramedullary rod;

FIG. 31 is another view showing navigation and placement of an intramedullary rod;

FIG. 32 is a screen face produced which assists in navigation and/or placement of an intramedullary rod;

FIG. 33 is another view of a screen face produced which assists in navigation and/or placement of an extramedullary rod.

FIG. 34 is a view which shows navigation and placement of an alignment guide;

FIG. 35 is a screen face which shows a fluoroscopic image of bone in combination with computer generated images of axes and components;

FIG. 36 is a view showing placement of a cutting block;

FIG. 37 is a view showing articulation of trial components during trial reduction; and

FIG. 38 is a screen face which may be used to assist in assessing joint function.

### DETAILED DESCRIPTION OF THE PREFERRED EMBODIMENTS

Various positional terms referring to the human anatomy—such as distal, proximal, medial, lateral, anterior and posterior—are used in this application in their customary and usual manner. The term “distal” refers to the area away from the point of attachment to the body, whereas the term “proximal” refers to the area near the point of attachment the body. The term “medial” refers to something situated closer to the middle of the body, while “lateral” refers to something situated closer to the left side or the right side of the body. Finally, “anterior” refers to something situated closer to the front of the body and “posterior” refers to something situated closer to the rear of the body.

Also, the term “mechanical axis” of the femur refers to an imaginary line drawn from the center of the femoral head to the center of the distal femur at the knee, and the term “anatomic axis” of the femur refers to an imaginary line drawn the middle of the femoral shaft. The angle between the mechanical axis and the anatomic axis is generally about six degrees.

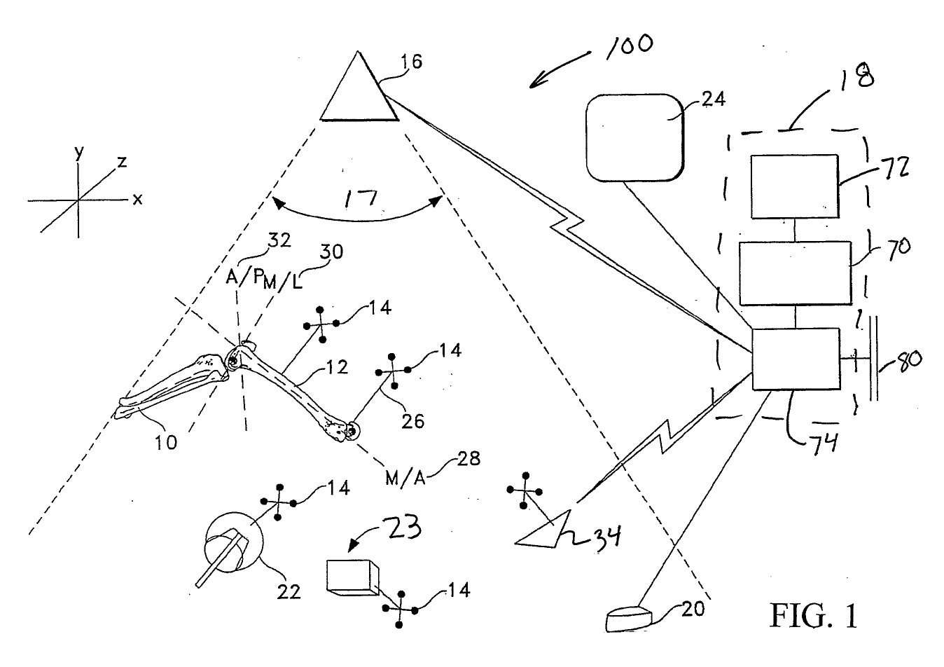

FIG. 1 is a schematic view showing one embodiment of a system 100 and one version of a setting in which surgery on a knee, in this case a Total Knee Arthroplasty, may be performed. The system 100 can track various body parts, such as tibia 10 and femur 12, to which fiducials 14 may be implanted, attached, or otherwise associated, be it physically, virtually, or otherwise. Fiducials 14 are structural frames that can be sensed by one or more sensors 16 suitable for sensing, storing, processing and/or outputting data (“tracking”) relating to position and orientation of fiducials 14 and, thus, components, such as tibia 10 and femur 12, that are attached or otherwise associated with the particular fiducial. The fiducials 14 may have active elements, passive elements or both. For example, some fiducials may include reflective elements, some may include light emitting diode (LED) active elements, and some fiducials include both reflective elements and active LED elements. Position/orientation sensor 16 may be any sort of sensor functionality for sensing position and orientation of fiducials 14 and, therefore, items that are associated, according to whatever desired electrical, magnetic, electromagnetic, sound, physical, radio frequency, or other active or passive technique. In the embodiment depicted in FIG. 1, position sensor 16 is a pair of infrared sensors or a stereoscopic infrared sensor disposed on the order of about one meter (sometimes more, sometimes less) apart and whose output can be processed in concert to provide position and orientation information regarding fiducials 14.

In the embodiment shown in FIG. 1, computing functionality 18 can include processing functionality 70, memory functionality 72, input/output functionality 74, whether on a standalone or distributed basis, via any desired standard, architecture, interface and/or network topology. Computing functionality 18 may be a stand alone computer, a networked computer, a mobile computing device, or similar device. In the case of a networked computer, the computing functionality 18 is connected to a network 80. In the depicted embodiment, computing functionality 18 is connected to a monitor 24 on which graphics and data may be presented to the surgeon during surgery. The monitor 24 may have a tactile interface so that the surgeon may point and click on screen for tactile screen input in addition to or instead of, if desired, keyboard and mouse conventional interfaces. Optionally, a foot pedal 20 or other convenient interface may be coupled to computing functionality 18 as can any other wireless or wired interface to allow the surgeon, nurse or other desired user to control or direct functionality 18 in order to, among other things, capture position/orientation information when certain components are oriented or aligned properly.

Item 22, such as trial components and prosthetic devices, instrument 23, or other devices used in a surgical procedure may be tracked in position and orientation by the sensor 16. For example, item 22 and instrument 23 may be tracked relative to tibia 10 and femur 12 using fiducials 14. As another example, item 22 and instrument 23 may be tracked relative to a global coordinate system.

Computing functionality 18 can process and store various forms of data. Further, computing functionality 18 can output data on touch-screen or monitor 24. As an example, the data may correspond in whole or in part to body parts or components, such as tibia 10, femur 12, or item 22. For example, in the embodiment shown in FIG. 1, tibia 10 and femur 12 are shown in cross-section or at least various internal aspects of them such as bone canals and surface structure are shown using fluoroscopic images. These images may be obtained using, as an example, a C-arm or imager attached to a fiducial 14. The body parts, for example, tibia 10 and femur 12, also have fiducials attached. When the fluoroscopy images are obtained using the C-arm with fiducial 14, a position/orientation sensor 16 “sees” and tracks the position of the fluoroscopy head as well as the positions and orientations of the tibia 10 and femur 12. The computer 18 stores the fluoroscopic images with this position/orientation information, thus correlating position and orientation of the fluoroscopic image relative to the relevant body part or parts. Thus, when the tibia 10 and corresponding fiducial 14 move, the computer 18 automatically and correspondingly senses the new position of tibia 10 in space and can correspondingly move implements, instruments, references, trials and/or implants on the monitor 24 relative to the image of tibia 10. Similarly, the image of the body part can be moved, both the body part and such items may be moved, or the on-screen image may otherwise be presented to suit the preferences of the surgeon or others and carry out the imaging that is desired. Similarly, when an item 22, such as an extramedullary rod, intramedullary rod, or other type of rod, that is being tracked moves, its image moves on monitor 24 so that the monitor shows the item 22 in proper position and orientation on monitor 24 relative to the femur 12. The item 22 can thus appear on the monitor 24 in proper or improper alignment with respect to the mechanical axis and other features of the femur 12, as if the surgeon were able to see into the body in order to properly navigate and position item 22. The computer functionality 18 can also store data relating to configuration, size and other properties of items 22, such as implements, instrumentation, trial components, implant components and other items used in surgery. When those are introduced into the field of position/orientation sensor 16, computer functionality 18 can generate and display overlaid or in combination with the fluoroscopic images of the body parts, such as tibia 10 and femur 12, computer generated images of implements, instrumentation components, trial components, implant components and other items for navigation, positioning, assessment and other uses.

In some embodiments, the system 100 may include a designator or probe 26. The probe 26 may be used in conjunction with the computer functionality 18 to track any point in a field 17 of the position/orientation sensor 16. One of the fiducials 14 is attached to probe 26 for tracking purposes. The surgeon, nurse, or other user touches the tip of probe 26 to a point such as a landmark on bone structure and actuates the foot pedal 20 or otherwise instructs the computer 18 to note the landmark position. The position/orientation sensor 16 “sees” the position and orientation of fiducial 14, “knows” where the tip of probe 26 is relative to that fiducial 14, and calculates and stores the point or other position designated by probe 26 when the foot pedal 20 is hit or other command is given to the computer 18. The computer 18 can also display on monitor 24 the identified point whenever desired and in whatever form or fashion or color. Thus, probe 26 can be used to designate landmarks on bone structure in order to allow the computer 18 to store and track, relative to movement of the fiducial 14, virtual or logical information, such as mechanical axis 28 of the femur 12, medial/lateral axis 30 and anterior/posterior axis 32 of femur 12, tibia 10 and other body parts in addition to any other virtual or actual construct or reference.

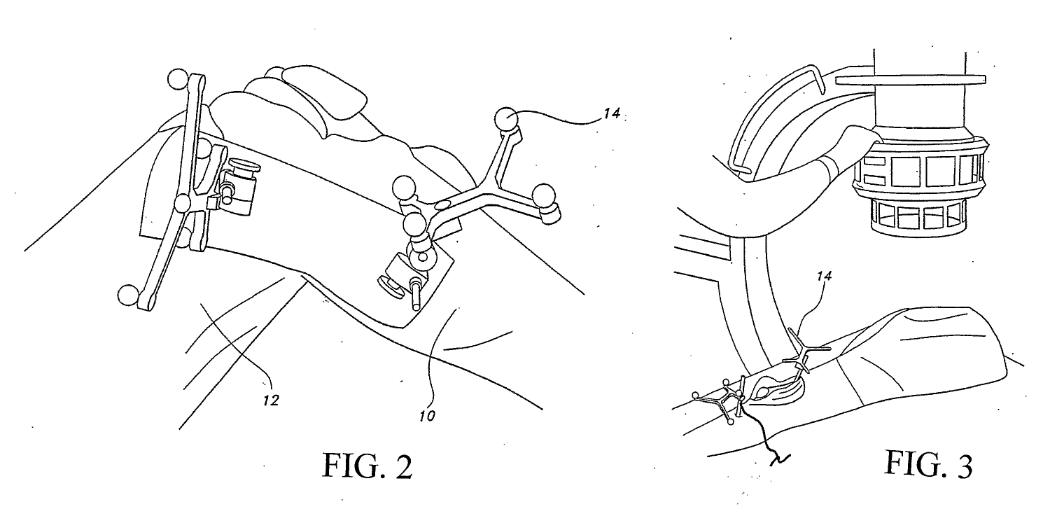

FIG. 2 shows a human knee in the surgical field, as well as the corresponding femur and tibia, to which fiducials 14 have been rigidly attached. In some embodiments, attachment of fiducials 14 is accomplished using structure that withstands vibration of surgical saws and other phenomenon which occur during surgery without allowing any substantial movement of fiducial 14 relative to body part being tracked by the system 100.

FIG. 3 shows fluoroscopy images being obtained of the body parts with fiducials 14 attached. The fiducial 14 on the fluoroscopy head in this embodiment is a cylindrically shaped cage which contains LEDs or “active” emitters for tracking by the sensors 16 (not shown in FIG. 3). Fiducials 14 attached to tibia 10 and femur 12 can also be seen. The fiducial 14 attached to the femur 12 uses LEDs instead of reflective spheres and is fed power by the wire seen extending into the bottom of the image.

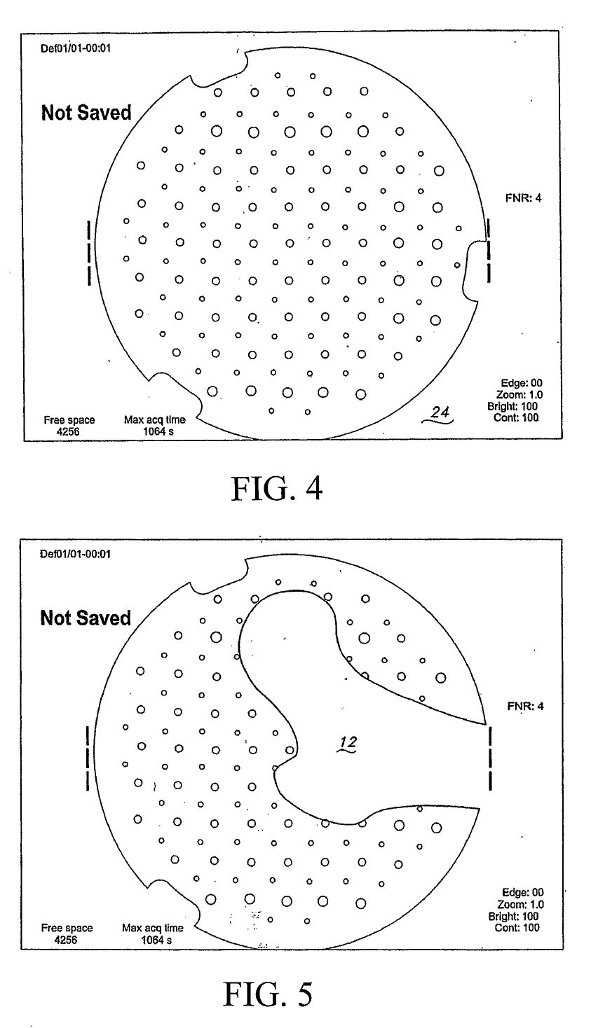

FIGS. 4-6 are fluoroscopic images shown on monitor 24 obtained with position and/or orientation information received by, noted and stored within computer 18. FIG. 4 is an open field with no body part image, but which shows the optical indicia which may be used to normalize the image obtained using a spherical fluoroscopy wave front with the substantially flat surface of the monitor 24. FIG. 5 shows an image of the femur 12 head. This image is taken in order to allow the surgeon to designate the center of rotation of the femoral head for purposes of establishing the mechanical axis and other relevant constructs relating to of the femur according to which the prosthetic components will ultimately be positioned. Such center of rotation can be established by articulating the femur within the acetabulum or a prosthesis to capture a number of samples of position and orientation information and in turn to allow the computer to calculate the average center of rotation. The center of rotation can be established by using the probe and designating a number of points on the femoral head and thus allowing the computer to calculate the geometrical center or a center which corresponds to the geometry of points collected. Additionally, graphical representations such as controllably sized circles displayed on the monitor can be fitted by the surgeon to the shape of the femoral head on planar images using tactile input on screen to designate the centers according to that graphic, such as are represented by the computer as intersection of axes of the circles. Those skilled in the art would understand that other techniques for determining, calculating or establishing points or constructs in space, whether or not corresponding to bone structure, may be used.

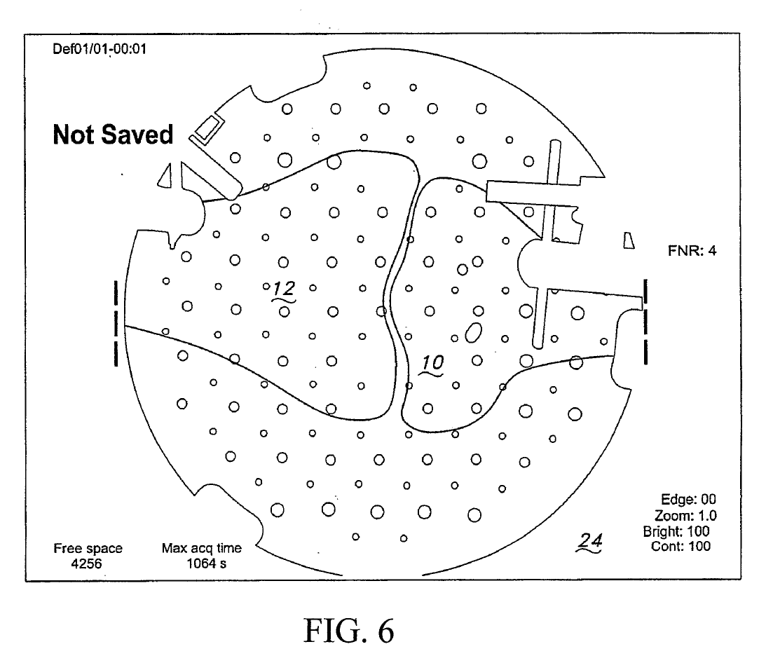

FIG. 5 shows a fluoroscopic image of the femoral head, while FIG. 6 shows an anterior/posterior view of the knee which can be used to designate landmarks and establish axes or constructs such as the mechanical axis or other rotational axes.

### Registration of Surgically Related Items

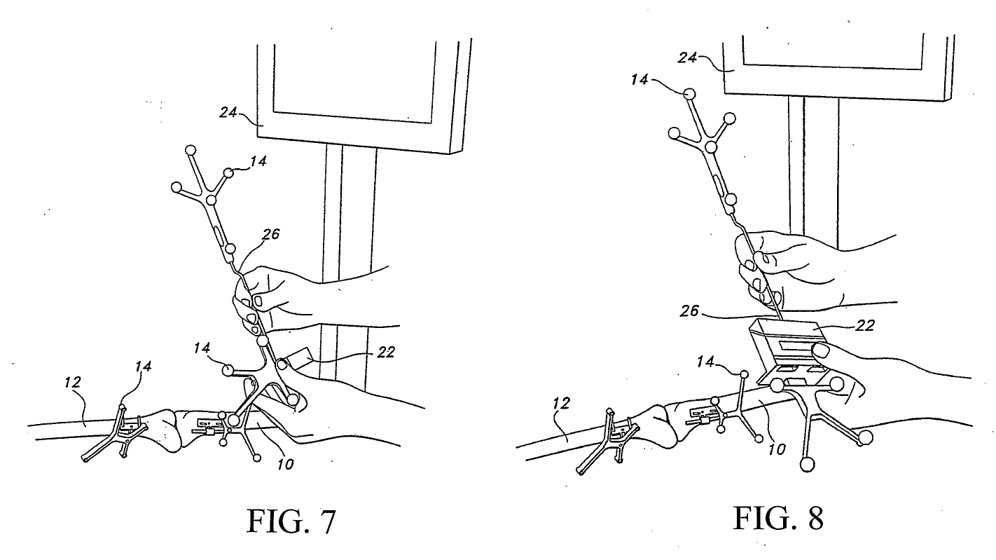

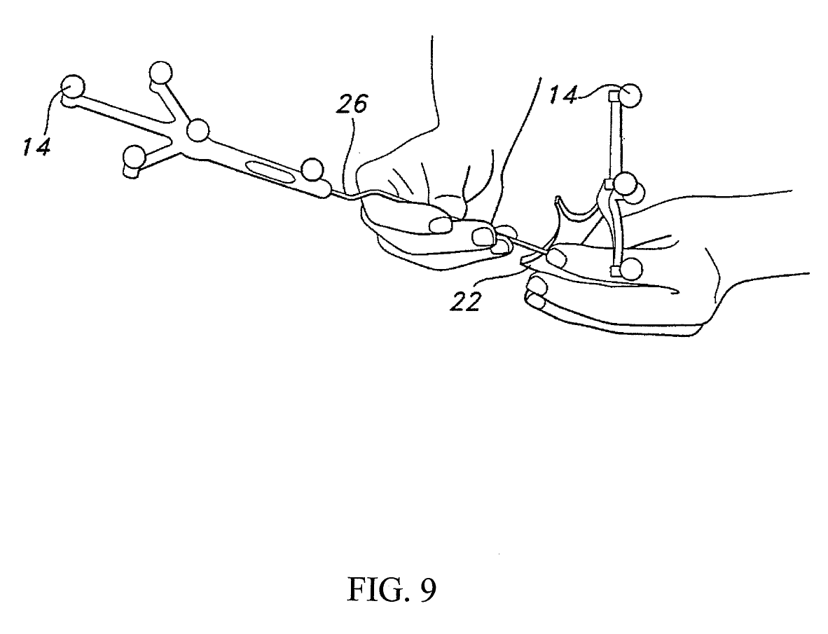

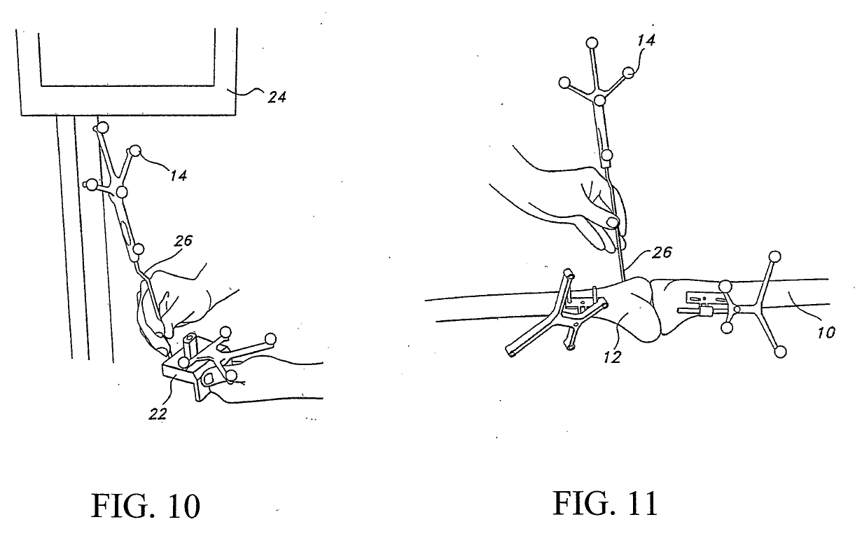

FIGS. 7-10 show designation or registration of items 22 which will be used in surgery. Registration simply means, however it is accomplished, ensuring that the computer 18 knows which body part, item or construct corresponds to which fiducial or fiducials 14, and how the position and orientation of the body part, item or construct is related to the position and orientation of its corresponding fiducial or a fiducial attached to an impactor or other component which is in turn attached to an item. Such registration or designation can be done before, after, or instead of registering bone or body parts as discussed with respect to FIGS. 4-6. FIG. 7 shows a technician designating with probe 26 an item 22, such as an instrument component to which fiducial 14 is attached. The sensor 16 “sees” the position and orientation of the fiducial 14 attached to the item 22 and also the position and orientation of the fiducial 14 attached to the probe 26 whose tip is touching a landmark on the item 22. The technician designates onscreen or otherwise the identification of the item and then activates the foot pedal or otherwise instructs the computer 18 to correlate the data corresponding to such identification, such as data needed to represent a particular cutting block component for a particular knee implant product, with the particularly shaped fiducial 14 attached to the component 22. The computer 18 has then stored identification, position and orientation information relating to the fiducial for component or item 22 correlated with the data such as configuration and shape data for the item 22 so that upon registration, when sensor 16 tracks the item 22 fiducial 14 in the infrared field, monitor 24 can show the cutting block component moving and turning, and properly positioned and oriented relative to the body part which is also being tracked. FIGS. 8-10 show similar registration for other instrumentation components 22.

### Registration of Anatomy and Constructs

Similarly, the mechanical axis and other axes or constructs of body parts 10 and 12 can also be “registered” for tracking by the system 100. As an optional step, the system 100 may employ a fluoroscope to obtain images of the femoral head, knee and ankle of the sort shown in FIGS. 4-6. The system 100 correlates such images with the position and orientation of the C-arm and the patient anatomy in real time as discussed above with the use of fiducials 14 placed on the body parts before image acquisition and which remain in position during the surgical procedure. Using these images, the surgeon can select and register in the computer 18 the center of the femoral head and ankle in orthogonal views, usually anterior/posterior and lateral, on a touch screen.

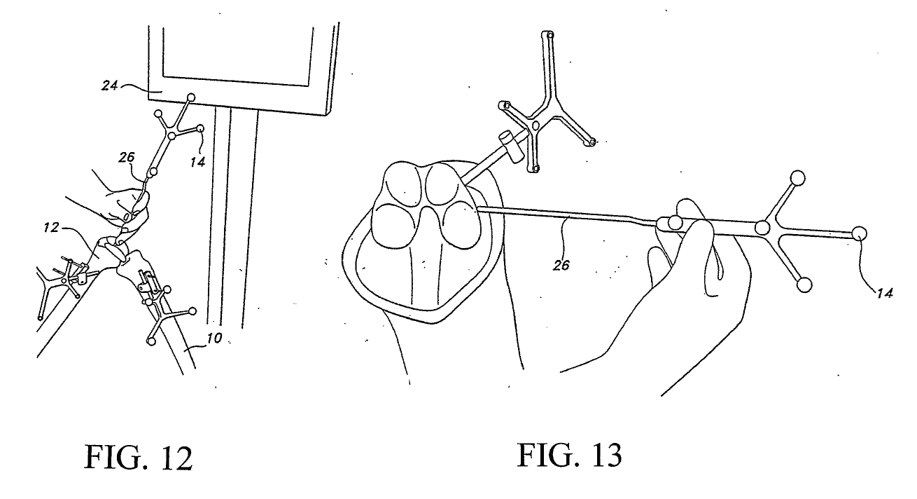

Alternatively, the surgeon or other person uses the probe 26 to select any desired anatomical landmarks or references to register body parts and related constructs. These points are registered in three dimensional space by the system 100 and are tracked relative to the fiducials 14 on the patient anatomy which are preferably placed intraoperatively. FIG. 11 shows the surgeon using probe 26 to designate or register landmarks on the condylar portion of femur 12 using probe 26 in order to feed to the computer 18 the position of one point needed to determine, store, and display the epicondylar axis. (See FIG. 16 which shows the epicondylar axis and the anterior-posterior plane and for lateral plane.) Although registering points using actual bone structure such as in FIG. 11 is one way to establish the axis, a cloud of points approach by which the probe 26 is used to designate multiple points on the surface of the bone structure can be employed, as can moving the body part and tracking movement to establish a center of rotation as discussed above. Once the center of rotation for the femoral head and the condylar component have been registered, the computer 18 is able to calculate, store, and render, and otherwise use data for, the mechanical axis 28 of the femur 12. FIGS. 12 and 13 once again show the probe 26 being used to designate points on the condylar component of the femur 12.

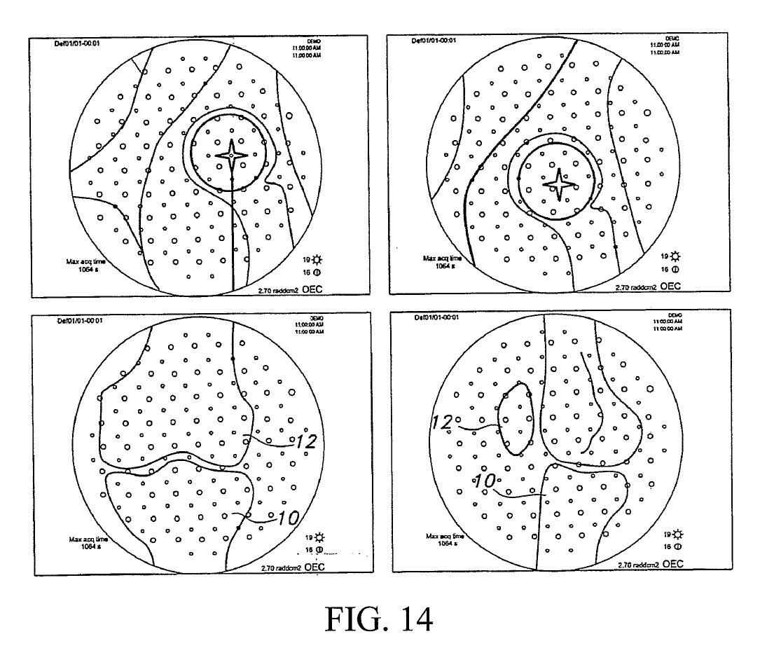

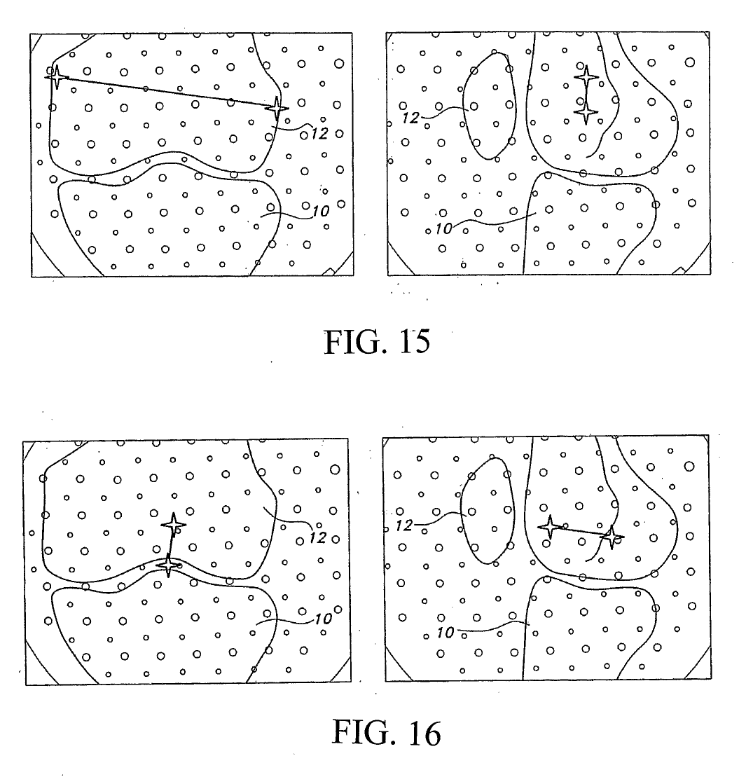

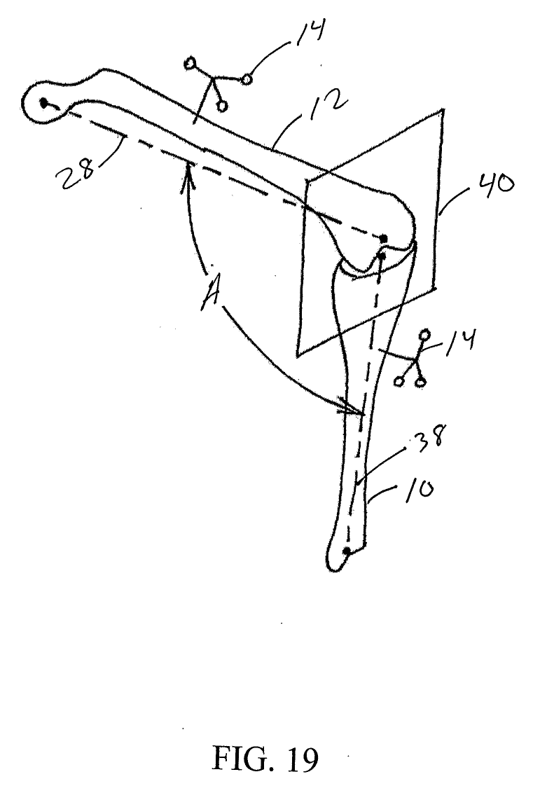

FIG. 14 shows the onscreen images being obtained when the surgeon registers certain points on the bone surface using the probe 26 in order to establish the femoral mechanical axis 28. Tibial mechanical axis 38 (best seen in FIG. 19) is then established by designating points to determine the centers of the proximal and distal ends of the tibia so that the mechanical axis can be calculated, stored, and subsequently used by the computer 18. FIG. 15 shows designated points for determining the epicondylar axis, both in the anterior/posterior and lateral planes, while FIG. 16 shows such determination of the anterior-posterior axis as rendered onscreen. The posterior condylar axis is also determined by designating points or as otherwise desired, as rendered on the computer generated geometric images overlain or displayed in combination with the fluoroscopic images, all of which are keyed to fiducials 14 being tracked by sensors 16.

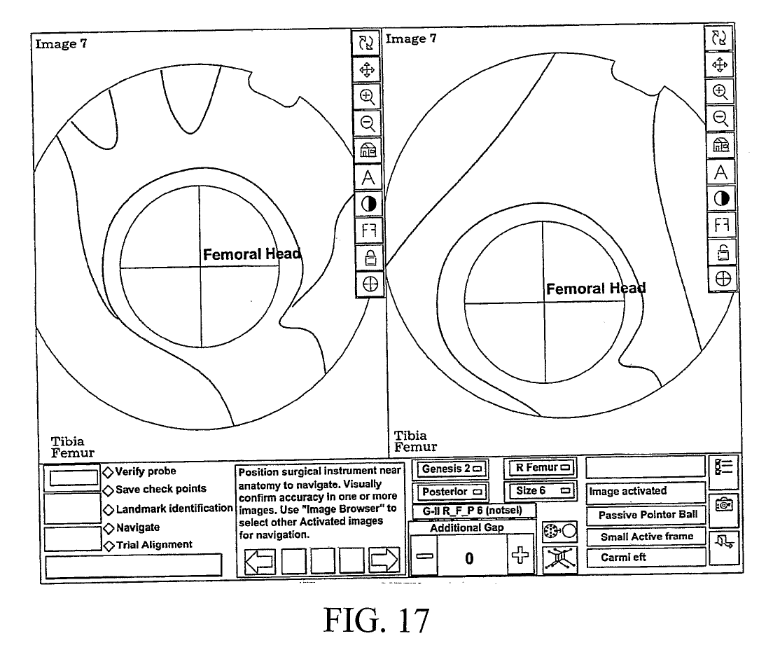

FIG. 17 shows an adjustable circle graphic which can be generated and presented in combination with orthogonal fluoroscopic images of the femoral head, and tracked by the computer 18 when the surgeon moves it on screen in order to establish the centers of the femoral head in both the anterior-posterior and lateral planes.

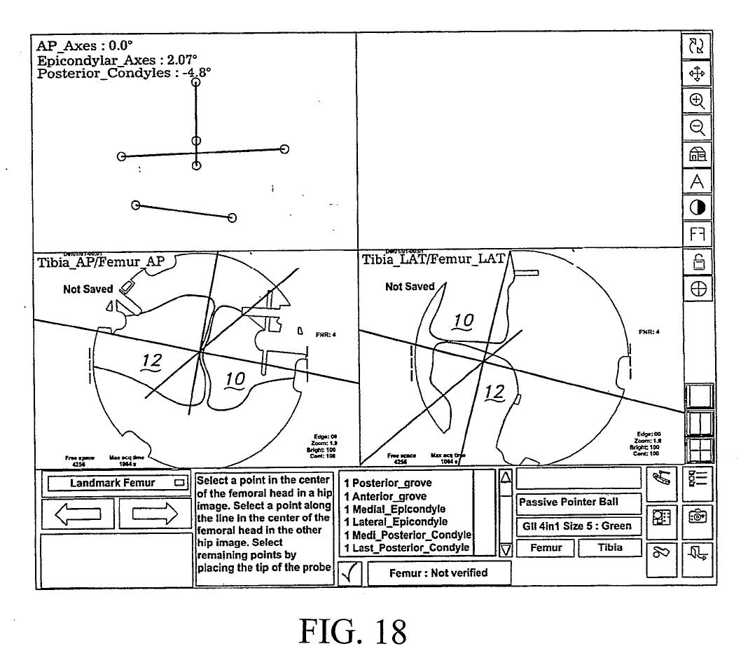

FIG. 18 is an onscreen image showing the anterior-posterior axis, epicondylar axis and posterior condylar axis from points which have been designated as described above. These constructs are generated by the computer 18 and presented on monitor 24. Optionally, the constructs may be presented in combination with the fluoroscopic images of the femur 12, correctly positioned and oriented relative thereto as tracked by the system 100. In the fluoroscopic/computer generated image combination shown at left bottom of FIG. 18, a “sawbones” knee as shown in certain drawings above which contains radio opaque materials is represented fluoroscopically and tracked using sensor 16 while the computer generates and displays the mechanical axis 28 of the femur 12, which runs generally horizontally. The epicondylar axis runs generally vertically, and the anterior/posterior axis runs generally diagonally. The image at bottom right shows similar information in a lateral view. Here, the anterior-posterior axis runs generally horizontally while the epicondylar axis runs generally diagonally, and the mechanical axis generally vertically.

FIG. 18, as is the case with a number of screen presentations, also shows at center a list of landmarks to be registered in order to generate relevant axes and constructs useful in navigation, positioning and assessment during surgery. Textural cues may also be presented which suggest to the surgeon next steps in the process of registering landmarks and establishing relevant axes. Such instructions may be generated as the computer 18 tracks, from one step to the next, registration of items 22 and bone locations as well as other measures being taken by the surgeon during the surgical operation.

FIG. 19 is a schematic view of a patient's leg with fiducials 14 associated therewith. In the embodiment depicted in FIG. 19, the tibia 10 is in flexion with respect to the femur 12. The femur 12 has a mechanical axis 28, and the tibia has a mechanical axis 38. Because the tibia 10 is in flexion, the femoral mechanical axis 28 is at an angle A relative to the tibial mechanical axis 38. In the embodiment depicted in FIG. 19, the angle A is about 90 degrees, plus or minus one degree. By tracking the femoral mechanical axis 28 and the tibial mechanical axis 38, the computing functionality 18 can identify when the axes are orthogonal to one another. The computing functionality 18 can then use this information to construct a tibial rotational plane 40 that extends through the tibial mechanical axis 38 and is substantially perpendicular to femoral mechanical axis 28. Thereafter, computing functionality 18 can use the constructed plane 40 to measure the angular rotation of items 22 about tibial mechanical axis 38. Alternatively, computing functionality 18 may use the constructed plane 40 to create a tibial coordinate system which includes the tibial mechanical axis 38, an anteroposterior axis and a medial-lateral axis. The medial-lateral axis, or transverse axis, is co-planar with the constructed plane 40 and orthogonal to the tibial mechanical axis 38, and the anteroposterior axis is orthogonal to both the constructed plane 40 and the tibial mechanical axis 38. Thereafter, the tibial coordinate system can be compared to other fiducials or a global coordinate system, and further, the tibial coordinate system can be used to identify orientation or position data of a surgical device, such as item 22, or construct, such as the femoral mechanical axis 28.

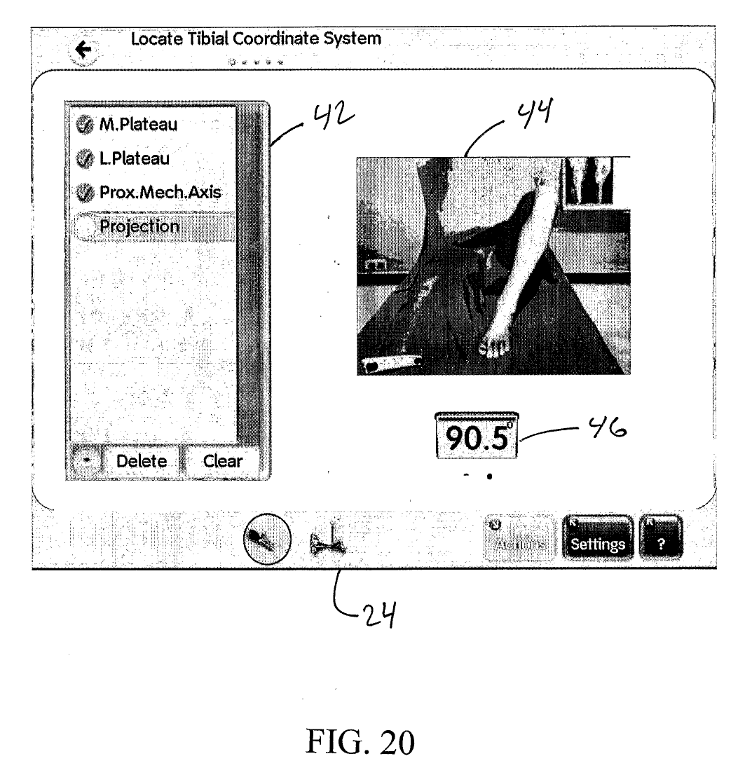

FIG. 20 illustrates the monitor 24 displaying degrees of flexion. The monitor 24 includes a first area 42 to display a menu, a second area 44 to display rendered images, and a third area 46 to display the amount of flexion between the femur 12 and the tibia 10. During construction of the tibial rotational plane, a user moves the tibia 10 relative to the femur 12 until the third area 46 displays about 90 degrees. Thereafter, the user indicates to the computer functionality 18 that the patient's knee is in the required amount of flexion. This indication may be accomplished by touching the monitor 24, by holding the knee in flexion for a predetermined period of time, through the use of the probe 26, or the through the use of the foot pedal 20.

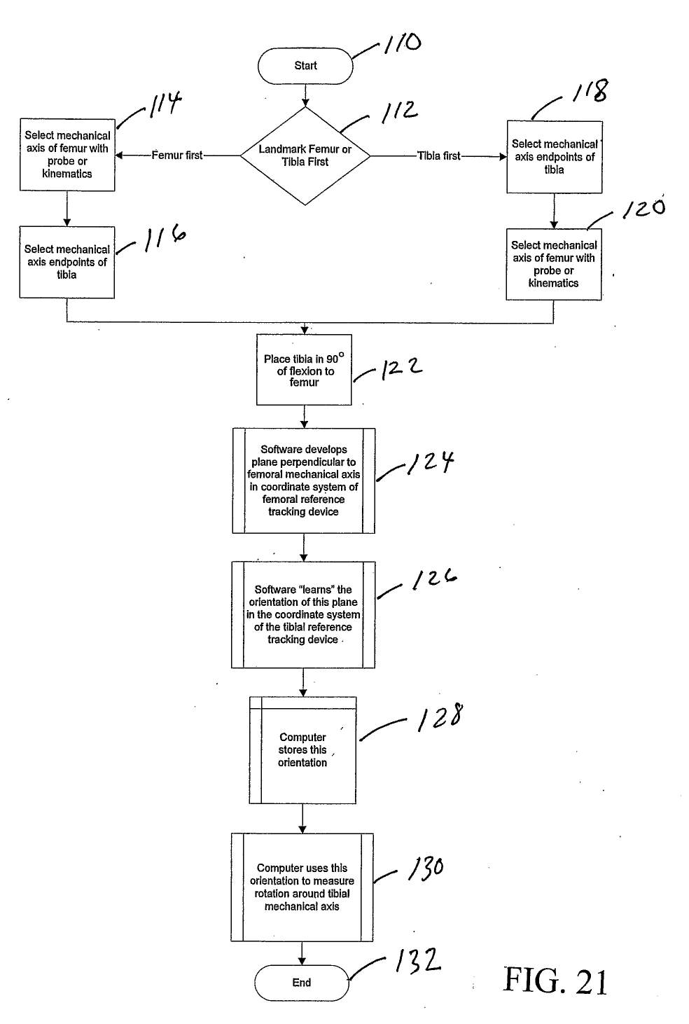

FIG. 21 illustrates the steps taken by the computing functionality 18 to create and use the tibial rotational plane 40. The computing functionality 18 begins at step 110. This may be a result of another software routine or a menu selection by a user. In step 112, a decision is made whether to start with the femur 12 or with the tibia 10. This step may be optional as some embodiments may specify that it is always best to start first with the femur and the tibia second, or vice versa. In steps 114 and 120, the femoral mechanical axis 28 is established. This may be done kinematically, through the use of fluoroscopic images, through the use of the probe 26 to identify landmarks of the femur, or some combination thereof. In steps 116 and 118, the tibial mechanical axis 38 is established by indicating landmarks of the tibia with the probe or through the use of fluoroscopic images. In step 122, the tibia 10 is placed in about 90 degrees of flexion relative to the femur 12. This places the tibial mechanical axis 38 substantially perpendicular to the femoral mechanical axis 28. The computing functionality 18 develops the tibial rotational plane 40 as extending through the tibial mechanical axis 38 and perpendicular to the femoral mechanical axis 28 in step 124. In step 126, the computing functionality 18 identifies the orientation of the tibial rotational plane 40 relative to fiducials 14 and/or relative to a global coordinate system. Computing functionality 18 stores this orientation into memory in step 128. Thereafter, computing functionality 18 can use the tibial rotational plane 40 as a reference to compare the angular rotation, orientation, or position of items 22 relative to the tibial mechanical axis 38 or to the tibial coordinate system described above. In FIG. 25, computing functionality 18 performs the angular comparison in step 130. However, those skilled in the art would understand that the steps necessary to establish the reference plane 40 and the comparison step 130 may be performed separately or together. For example, the reference plane 40 first may be established and at a later time, such as by menu selection, the comparison step 130 is performed. After the reference plane 40 is stored in memory, the routine ends in step 132.

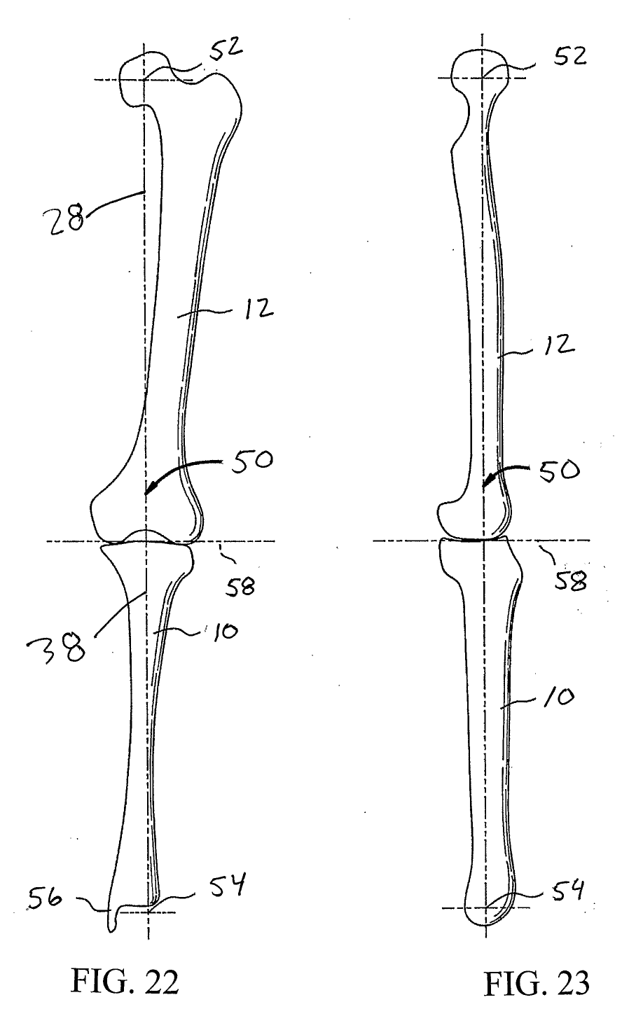

FIGS. 22 and 23 show in schematic form the relationship of the weight bearing axis (WBA) 50 to a left human femur 12 and tibia 10 in normal stance. FIG. 22 is a schematic in the coronal (medial-lateral) plane of the patient and FIG. 23 is in the sagital (anterior-posterior) plane of the patient. Weight bearing axis 50 is defined to pass through two points: the center of the hip joint 52 and the center of the ankle joint 54. Weight bearing axis 50 normally passes slightly medial to the anatomic center of the knee joint although this may very considerably from patient to patient. Hip joint center 52 is defined as the center of rotation of the hip joint and is generally accepted to be the anatomic center of the head of the femur. Ankle joint center 54 is defined as the center of rotation of the ankle joint and is generally accepted to lie midway along an axis passing through the malleoli of the lower limb. Medial malleolus 56 exists on the distal end of the tibia 10. The lateral malloelus is a similar structure on the distal end of the fibula (not shown). Joint line 58 is a plane perpendicular to weight bearing axis 50 at a point approximating the bearing surface between femur 12 and tibia 10.

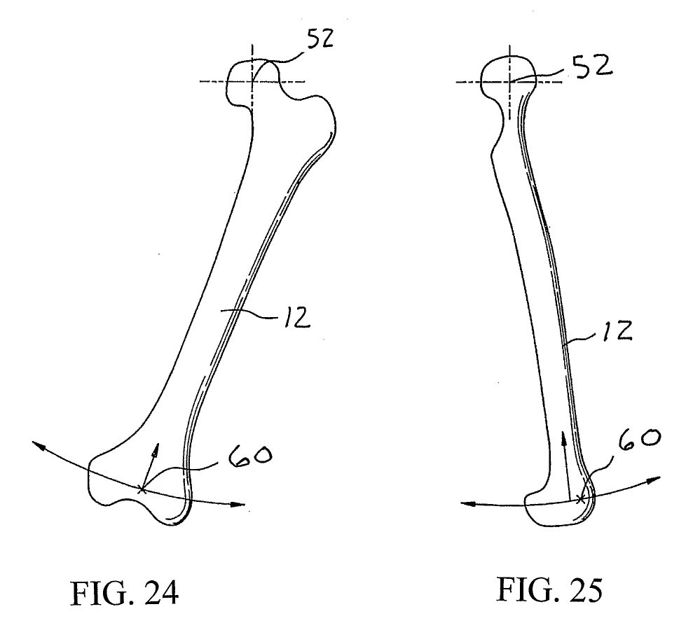

FIGS. 24 and 25 show in schematic form the motion of femur 12 about hip joint center 52 in the patient's coronal and sagital planes respectively. The motion of femur 12 is governed by the ball socket hip joint such that, during any movement of femur 12, femoral registration point 60 fixed with respect to femur 12 will be constrained to move on the surface of a theoretical sphere with center at hip joint center 52 and radius equal to the distance between femoral registration point 60 and hip joint center 52. By measuring the vectorial displacement between three or more successive positions of femoral registration point 60 in a reference frame in which hip joint center 52 remains stationary as femur 12 is moved, the position of hip joint center 52 in that reference frame can be calculated. Additionally, the location of hip joint center 52 with respect to femoral registration point 60 can also be calculated. Increasing the number of measured positions of femoral registration point 60 increases the accuracy of the calculated position of hip joint center 52. By using the probe 26 to locate registration points 60, the computer 18 can calculate the geometrical center or a center which corresponds to the geometry of points collected.

Other methods may be used to identify the hip joint center 52. For example, The femoral head may be located using various scanning techniques, such as computed tomography (CT) or magnetic resonance imaging (MRI). Further, the hip joint center 52 may be located through laser triangulation. The laser method is similar to measuring the vectorial displacement. A laser is mounted on the distal end of the femur, and the femur is rotated in the acetabulum or a prosthesis to capture a number of samples of position and orientation information. The laser light indicates the center of rotation on a target, which is used by the laser operator to identify the center of the femoral head.

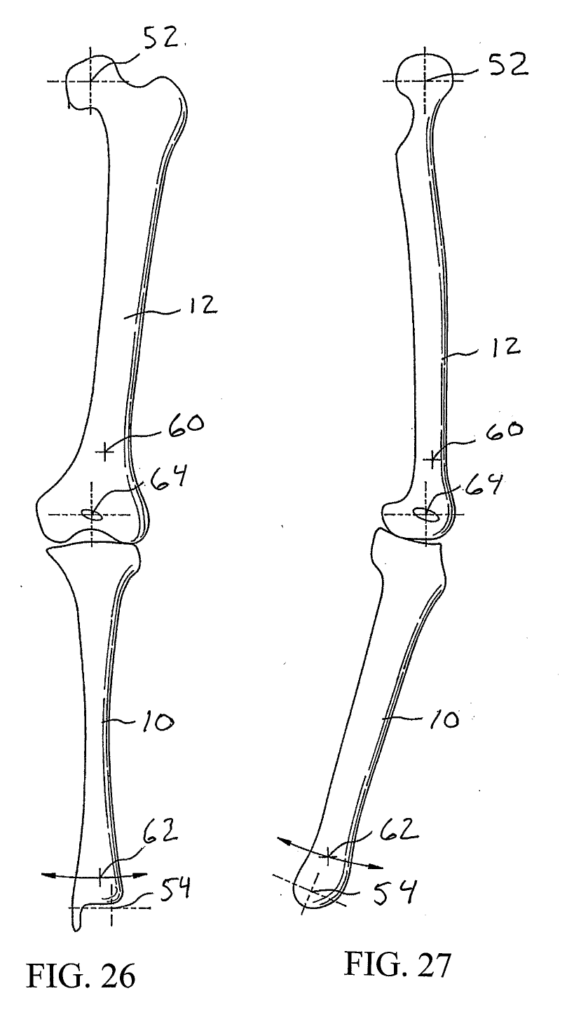

FIGS. 26 and 27 show in schematic form a simplified representation of the motion of tibia 10 with respect to femur 12 in the patient's coronal and sagital planes respectively. The motion of tibia 10 with respect to femur 12 is a complex, six degree-of-freedom relationship governed by the ligamentous tension and the three bearing surfaces of the knee joint. However for the purposes of implant location, a reasonable approximation of the motion of tibia 10 can be made assuming the knee joint to be a sliding hinge in the sagital plane with limited motion in the coronal plane. Based on these simplifying assumptions, movement of tibial registration point 62 fixed with respect to tibia 10 will be constrained to move on the surface of a theoretical sphere with instantaneous center within the locus of knee joint center 64 and radius equal to the distance between tibial registration point 62 and knee joint center 64. Because the bony nature of the human ankle permits intraoperative estimation of ankle joint center 54 by palpation, tibial registration point 62 can be fixed to tibia 10 at a known vectorial displacement from ankle joint center 64 through the use of a notched guide or boot strapped to the lower limb as is commonly known in knee arthroplasty. Measurement of the vectorial displacement of tibial registration point 62 with respect to femoral registration point 60, previously fixed-relative to femur 12 and at a calculated position relative to hip joint center 52, thereby permits the calculation of the vectorial position of ankle joint center 64 with respect to hip joint center 52 and the weight bearing axis to be determined. As with calculation of the position hip joint center 52, repeated measurements improve the accuracy of the determined weight bearing axis 50.

Further, by measuring the vectorial displacement between successive positions of tibial registration point 62 in a reference frame in which femoral registration point 60 remains stationary as tibia 10 is moved, the locus of positions of knee joint center 64 in that reference frame can be calculated.

By identifying the vectorial displacements, the hip joint center 52, and the ankle joint center 54, computing functionality 18 can “learn” and “memorize” the femoral mechanical axis 28 and the tibial mechanical axis 38. Thereafter, computing functionality 18 can construct the tibial reference plane 40.

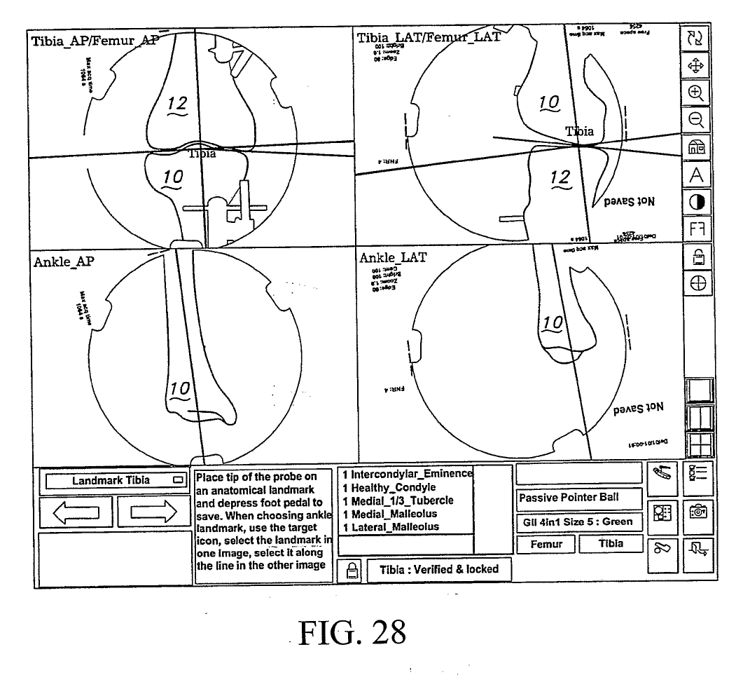

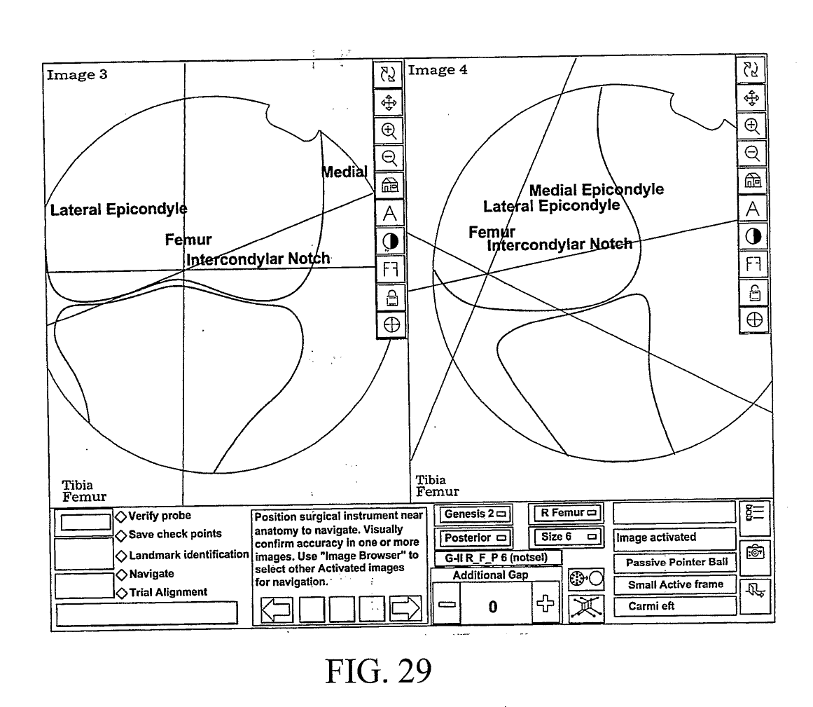

FIG. 28 shows mechanical, lateral, anterior-posterior axes for the tibia according to points registered by the surgeon. FIG. 29 is another onscreen image showing the axes for the femur 12.

### Modifying Bone

After the mechanical axis and other rotation axes and constructs relating to the femur and tibia are established, instrumentation can be properly oriented to resect or modify bone in order to properly fit trial components and implant components. Instrumentation such as, for instance, cutting blocks, to which fiducials 14 are mounted, can be employed. The system 100 can then track instrumentation as the surgeon manipulates it for optimum positioning. In other words, the surgeon can “navigate” the instrumentation for optimum positioning using the system and the monitor. In this manner, instrumentation may be positioned according to the system of this embodiment in order to align the ostetomies to the mechanical and rotational axes or reference axes and planes on a rod (extramedullary, intramedullary, or other type) that does not violate the canal. The monitor 24 also can then display the instrument, such as the cutting block and/or the implant relative to the instrument and the rod during this process, in order to, among other things, properly select implant size and perhaps implant type. As the instrument moves, the varus/valgus, flexion/extension and internal/external rotation of the relative component position can be calculated and shown with respect to the referenced axes; in some embodiments, this can be done at a rate of six cycles per second or faster. The instrument position is then fixed in the computer and physically, and the surgeon makes the bone resections.

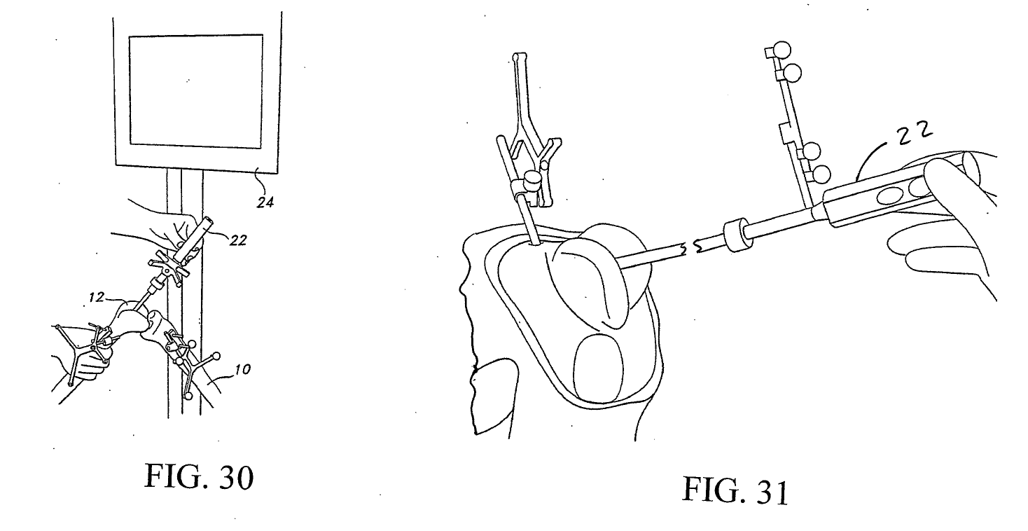

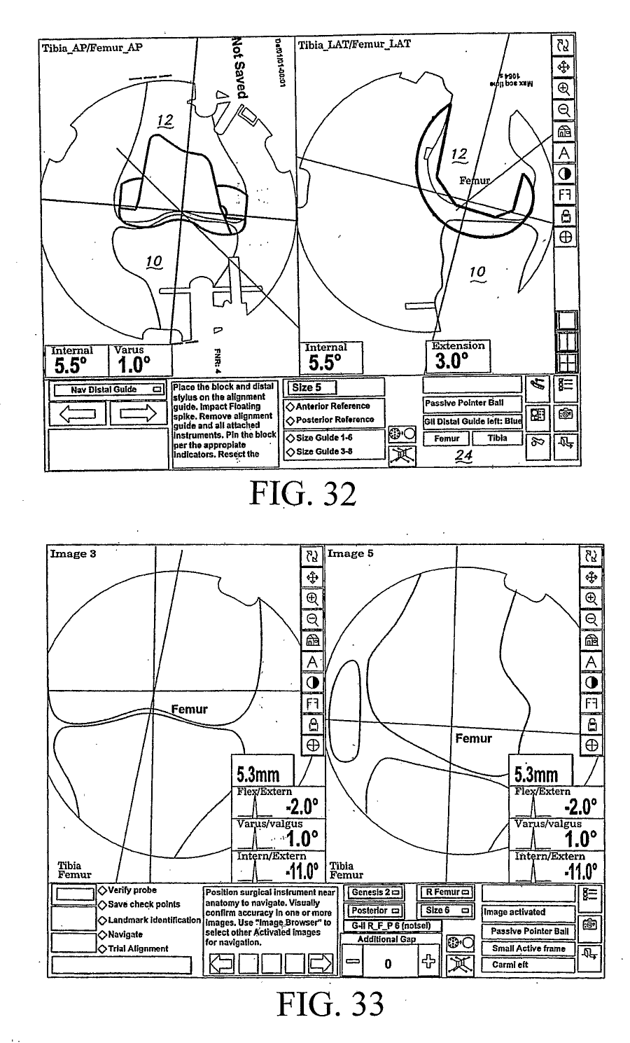

FIG. 30 shows orientation of an intramedullary rod to which a fiducial 14 is attached via item 22, such as an impactor. The surgeon views the monitor 24 which has an image as shown in FIG. 32 of the rod overlain on or in combination with a fluoroscopic image of the femur 12 as the two are actually positioned and oriented relative to one another in space. The surgeon then navigates the rod into place preferably along the mechanical axis of the femur and drives it home with appropriate mallet or other device. This may avoid the need to bore a hole in the metaphysis of the femur and place a reamer or other rod into the medullary canal, which can cause fat embolism, hemorrhaging, infection and other untoward and undesired effects.

FIG. 31 also shows the intramedullary rod being located. FIG. 32 shows fluoroscopic images, both anterior-posterior and lateral, with axes, and with a computer generated and tracked image of the rod superposed or in combination with the fluoroscopic images of the femur and tibia. FIG. 33 shows the rod superposed on the femoral fluoroscopic image similar to what is shown in FIG. 32.

FIG. 32 also shows other information relevant to the surgeon such as the name of the component being overlain on the femur image (new EM nail), suggestions or instructions at the lower left, and angle of the rod in varus/valgus and extension relative to the axes. Any or all of this information can be used to navigate and position the rod relative to the femur. At a point in time during or after placement of the rod, its tracking may be “handed off” from the impactor fiducial 14 to the femur fiducal 14 as discussed below.

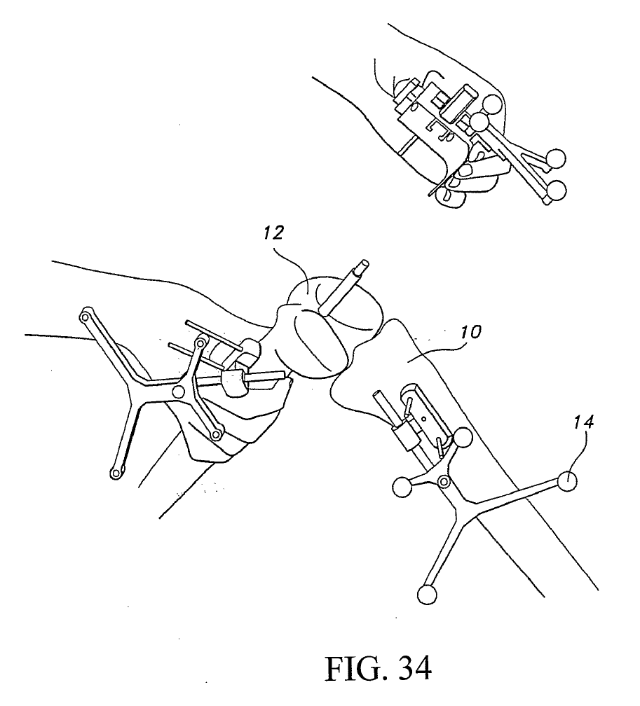

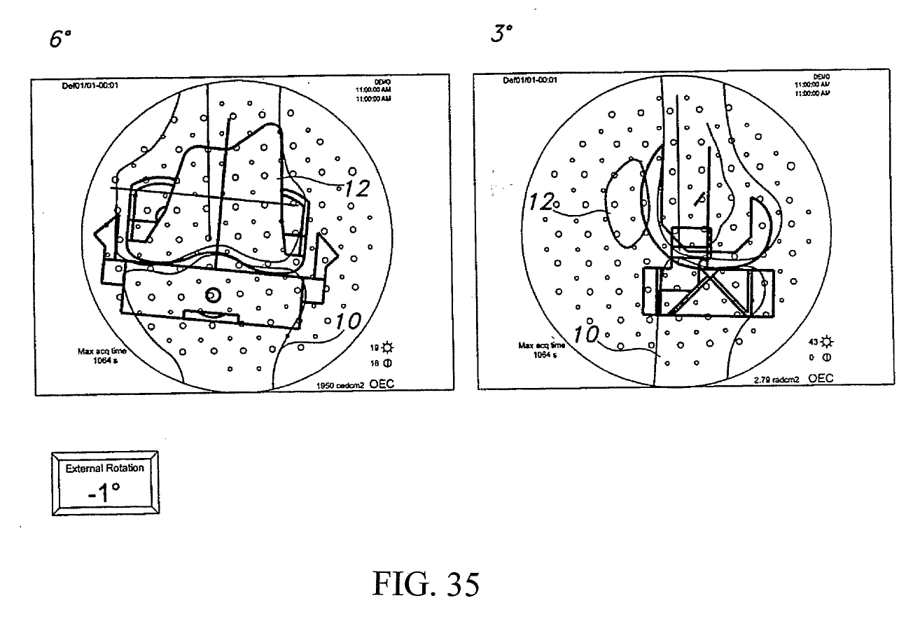

Once the extramedullary rod, intramedullary rod, other type of rod has been placed, instrumentation can be positioned as tracked in position and orientation by sensor 16 and displayed on screen face 24. Thus, a cutting block of the sort used to establish the condylar anterior cut, with its fiducial 14 attached, is introduced into the field and positioned on the rod. FIG. 34 illustrates a cutting block being positioned. Because the cutting block corresponds to a particular implant product and can be adjusted and designated on screen to correspond to a particular implant size of that product, the computer 18 can generate and display a graphic of the cutting block and the femoral component overlain on the fluoroscopic image as shown in FIG. 35. The surgeon can thus navigate and position the cutting block on screen using not only images of the cutting block on the bone, but also images of the corresponding femoral component that ultimately will be installed. The surgeon can adjust the positioning of the physical cutting block component and secure it to the rod in order to resect the anterior of the condylar portion of the femur in order to optimally fit and position the ultimate femoral component being shown on the screen. Other cutting blocks and other resections may be positioned and made similarly on the condylar component.

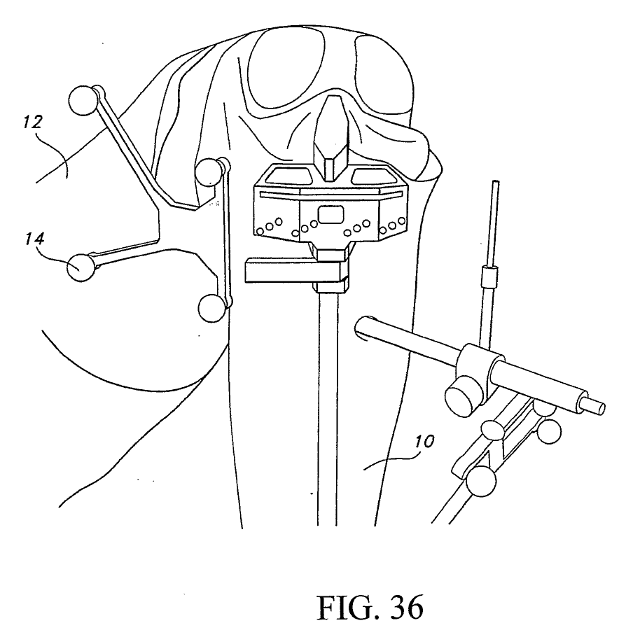

In a similar fashion, instrumentation may be navigated and positioned on the proximal portion of the tibia 10 as shown in FIG. 36 and as tracked by sensor 16 and on screen by images of the cutting block and the implant component as shown in FIG. 35.

In summary, the computer 18 and monitor 24 show femoral component and tibial component overlays according to certain position and orientation of cutting blocks/instrumentation as bone resections are made. The surgeon can thus visualize where the implant components will be and can assess fit, and other things if desired, before resections are made.

### Navigation, Placement and Assessment of Trials and Implants

Once resection and modification of bone has been accomplished, implant trials can then be installed and tracked by the system 100 in a manner similar to navigating and positioning the instrumentation, as displayed on the screen 24. Thus, a femoral component trial, a tibial plateau trial, and a bearing plate trial may be placed as navigated on screen using computer generated overlays corresponding to the trials.

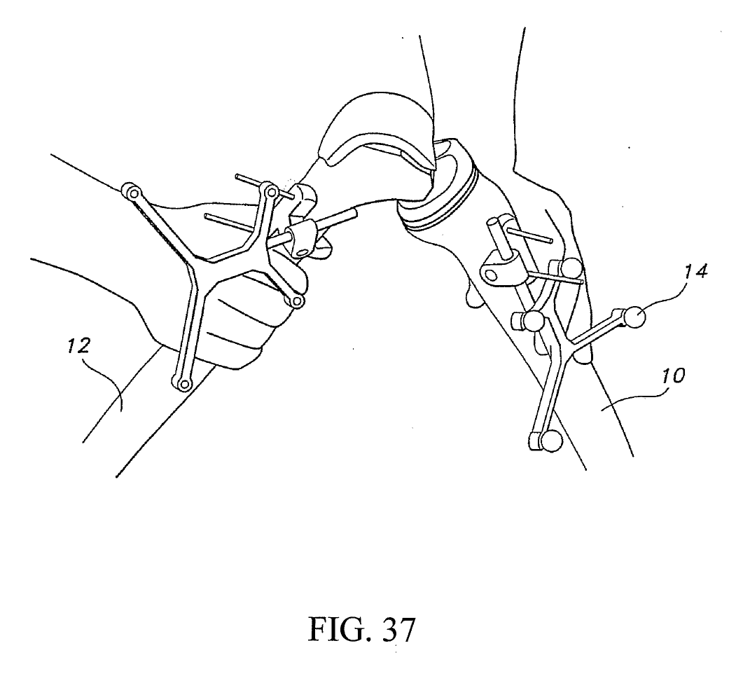

During the trial installation process, and also during the implant component installation process, instrument positioning process or at any other desired point in surgical or other operations, the system 100 can transition or segue from tracking a component according to a first fiducial to tracking the component according to a second fiducial. Thus, as shown as FIG. 37, the trial femoral component is mounted on an impactor to which is attached a fiducial 14. The trial component is installed and positioned using the impactor. The computer 18 “knows” the position and orientation of the trial relative to the fiducial on the impactor (such as by prior registration of the component attached to the impactor) so that it can generate and display the image of the femoral component trial on screen 24 overlaid on the fluoroscopic image of the condylar component. At any desired point in time, before, during or after the trial component is properly placed on the condylar component of the femur to align with mechanical axis and according to proper orientation relative to other axes, the system 100 can be instructed by foot pedal or otherwise to begin tracking the position of the trial component using the fiducial attached to the femur rather than the one attached to the impactor. The sensor 16 “sees” at this point in time both the fiducials on the impactor and the femur 12 so that it already “knows” the position and orientation of the trial component relative to the fiducial on the impactor and is thus able to calculate and store for later use the position and orientation of the trial component relative to the femur 12 fiducial. Once this “handoff” happens, the impactor can be removed and the trial component tracked with the femur fiducial 14 as part of or moving in concert with the femur 12. Similar handoff procedures may be used in any other instance as desired.

The tibial trial may be placed on the proximal tibia and then registered using the probe 26. Probe 26 is used to designate preferably at least three features on the tibial trial of known coordinates, such as bone spike holes. As the probe 26 is placed onto each feature, the system 100 is prompted to save that coordinate position so that the system 100 can match the tibial trial's feature's coordinates to the saved coordinates. The system 100 then tracks the tibial trial relative to the tibial anatomical reference frame.

Once the trial components are installed, the surgeon can assess alignment and stability of the components and the joint. During such assessment, in trial reduction, the computer can display on monitor 24 the relative motion between the trial components to allow the surgeon to make soft tissue releases and changes in order to improve the kinematics of the knee. The system 100 can also apply rules and/or intelligence to make suggestions based on the information such as what soft tissue releases to make if the surgeon desires. The system 100 can also display how the soft tissue releases are to be made.

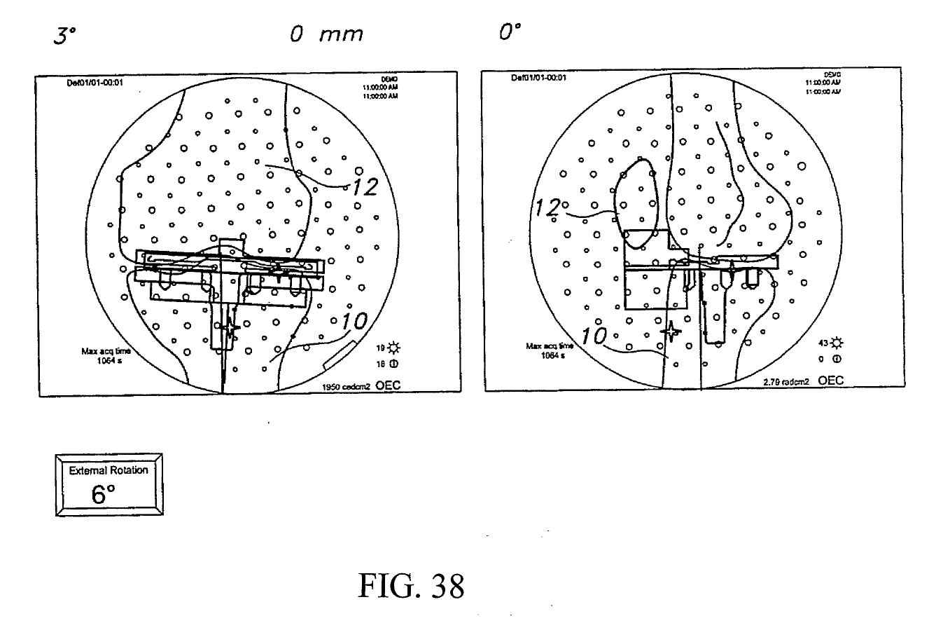

FIG. 37 shows the surgeon articulating the knee as he monitors the screen which is presenting images such as those shown in FIG. 38 which not only show movement of the trial components relative to each other, but also orientation, flexion, and varus/valgus data. During this assessment, the surgeon may conduct certain assessment processes such as external/internal rotation or rotational laxity testing, varus/valgus tests, and anterior-posterior drawer at 0 and 90 degrees and mid range. Thus, in the AP drawer test, the surgeon can position the tibia at the first location and press the foot pedal. The surgeon then positions the tibia at the second location and once again presses the foot pedal so that the computer has registered and stored two locations in order to calculate and display the drawer and whether it is acceptable for the patient and the product involved. If not, the computer can apply rules in order to generate and display suggestions for releasing ligaments or other tissue, or using other component sizes or types. Once the proper tissue releases have been made, if necessary, and alignment and stability are acceptable as noted quantitatively on screen about all axes, the trial components may be removed and actual components navigated, installed, and assessed in performance in a manner similar to that in which the trial components were navigated, installed, and assessed.

At the end of the case, all alignment information can be saved for the patient file. This is of great assistance to the surgeon due to the fact that the outcome of implant positioning can be seen before any resections have been made to the bone. The system 100 is also capable of tracking the patella and resulting placement of cutting guides and the patellar trial position. The system 100 then tracks alignment of the patella with the patellar femoral groove and will give feedback on issues, such as, patellar tilt.

The tracking and image information provided by the system 100 facilitate telemedical techniques because it provides useful images for distribution to distant geographic locations where expert surgical or medical specialists may collaborate during surgery. Thus, the system can be used in connection with computing functionality 18 which is networked or otherwise in communication with computing functionality in other locations, whether by public switched telephone network (PSTN), information exchange infrastructures, such as packet switched networks, including the Internet. Such remote imaging may occur on computers, wireless devices, videoconferencing devices or in any other mode or on any other platform which is now or may in the future be capable of rending images or parts of them. Parallel communication links, such as switched or unswitched telephone call connections, may also accompany or form part of such telemedical techniques. Distant databases, such as online catalogs of implant suppliers or prosthetics buyers or distributors, may form part of or be networked with functionality 18 to give the surgeon in real time access to additional options for implants which could be procured and used during the surgical operation.

The invention may include one or more of the following steps. An optional first step is to obtain appropriate images, such as fluoroscopy images of appropriate body parts. This first step may include tracking the imager via an associated fiducial whose position and orientation is tracked by position/orientation sensors, such as stereoscopic infrared (active or passive) sensors. A second step is to register tools, instrumentation, trial components, prosthetic components, and other items to be used in surgery. The second step may include associating the tool, instrument, trial component, prosthetic component, or other device with a corresponding fiducial. A third step is to locate and register body structure, such as designating points on the femur and tibia using a probe associated with a fiducial, in order to provide the processing functionality information relating to the body part, such as rotational axes. A fourth step is to navigate and position instrumentation, such as cutting instrumentation, in order to modify bone, at least partially using images generated by the processing functionality corresponding to what is being tracked and/or has been tracked, and/or is predicted by the system, and thereby resecting bone effectively, efficiently and accurately. A fifth step is to navigate and position trial components, such as femoral components and tibial components, some or all of which may be installed using impactors with a fiducial and, if desired, at the appropriate time discontinuing tracking the position and orientation of the trial component using the impactor fiducial and starting to track that position and orientation using the body part fiducial on which the component is installed. A sixth step is to assess alignment and stability of the trial components and joint, both statically and dynamically as desired, using images of the body parts in combination with images of the trial components while conducting appropriate rotation, anterior-posterior drawer and flexion/extension tests and automatically storing and calculating results to present data or information which allows the surgeon to assess alignment and stability. A seventh step includes the release of tissue, such as ligaments, if necessary and adjusting trial components as desired for acceptable alignment and stability. An eighth step includes installation of implant components whose positions may be tracked at first via fiducials associated with impactors for the components and then tracked via fiducials on the body parts in which the components are installed. A ninth step includes assessing alignment and stability of the implant components and joint by use of some or all tests mentioned above and/or other tests as desired, releasing tissue if desired, adjusting if desired, and otherwise verifying acceptable alignment, stability and performance of the prosthesis, both statically and dynamically. Some or all of these steps may be used in any total or partial joint repair, reconstruction or replacement, including knees, hips, shoulders, elbows, ankles and any other desired joint in the body.

The system uses computer capacity, including standalone and/or networked computer capacity, to store data regarding spatial aspects of surgically related items and virtual constructs or references including body parts, implements, instrumentation, trial components, prosthetic components and rotational axes of body parts. Any or all of these may be physically or virtually connected to or incorporate any desired form of mark, structure, component, or other fiducial or reference device or technique which allows position and/or orientation of the item to which it is attached to be sensed and tracked, preferably in three dimensions of translation and three degrees of rotation as well as in time if desired. As an example, such “fidicuals” are reference frames each containing at least three, preferably four, sometimes more, reflective elements, such as spheres reflective of lightwave or infrared energy, or active elements, such as light emitting diodes (LEDs).

In one embodiment, orientation of the elements on a particular fiducial varies from one fiducial to the next so that sensors may distinguish between various components to which the fiducials are attached in order to correlate for display and other purposes data files or images of the components. The fiducials may be active, passive, or some combination thereof. In other words, some fiducials use reflective elements and some use active elements, both of which may be tracked by preferably two, sometimes more infrared sensors whose output may be processed in concert to geometrically calculate position and orientation of the item to which the fiducial is attached.

Position/orientation tracking sensors and fiducials need not be confined to the infrared spectrum. Any electromagnetic, electrostatic, light, sound, radiofrequency or other desired technique may be used. Alternatively, each item, such as a surgical implement, instrumentation component, trial component, implant component or other device may contain its own “active” fiducial, such as a microchip with appropriate field sensing or position/orientation sensing functionality and communications link, such as spread spectrum radio frequency (RF) link, in order to report position and orientation of the item. Such active fiducials, or hybrid active/passive fiducials, such as transponders, can be implanted in the body parts or in any of the surgically related devices mentioned above or conveniently located at their surface or otherwise as desired. Fiducials may also take the form of conventional structures, such as a screw driven into a bone, or any other three dimensional item attached to another item, position and orientation of such three dimensional item able to be tracked in order to track position and orientation of body parts and surgically related items. Hybrid fiducials may be partly passive, partly active such as inductive components or transponders which respond with a certain signal or data set when queried by sensors.

The system employs a computer to calculate and store reference axes of body components, such as in a total knee arthroplasty, for example, the mechanical axis of the femur and tibia. From these axes such systems track the position of the instrumentation and osteotomy guides so that bone resections will locate the implant position optimally, usually aligned with the mechanical axis. Furthermore, during trial reduction of the knee, the system provides feedback on the balancing of the ligaments in a range of motion and under varus/valgus, anterior/posterior and rotary stresses and can suggest or at least provide more accurate information than in the past about which ligaments the surgeon should release in order to obtain correct balancing, alignment and stability. The system can also suggest modifications to implant size, positioning, and other techniques to achieve optimal kinematics. The system can also include databases of information regarding tasks such as ligament balancing, in order to provide suggestions to the surgeon based on performance of test results as automatically calculated by such systems and processes.

The invention also includes a computerized method for determining tibial rotation within a coordinate system. The method may include one or more of the following steps, which are provided in no particular order. A first step of the method is to provide a computer having a processor, a memory, and an input/output device. A second step is to identify a mechanical axis of a femur. A third step is to identify a mechanical axis of a tibia. A fourth step is to place the tibia in about 90 degrees of flexion relative to the femur. A fifth step is to construct a plane through the mechanical axis of the tibia and orthogonal to the mechanical axis of the femur. The constructed plane may be used to create a tibial coordinate system which includes the mechanical axis of the tibia, an anteroposterior axis and a medial-lateral axis. A sixth step is to identify an orientation of the plane relative to other fiducials or a global coordinate system. A seventh step is to store the orientation of the plane in the memory of the computer. An eighth step is to measure an angular rotation of an item relative to the plane and the mechanical axis of the tibia or to the tibial coordinate system. Items may include, but are not limited to, tools, instruments, trial components, and prosthetic devices. The step of identifying a mechanical axis of a femur may include the step of locating data points corresponding to structure of the femur. The step of identifying a mechanical axis of a tibia may include the step of locating data points corresponding to structure of the tibia.

The invention may also include one or more of the following optional steps. For example, the method may include the step of storing in the memory the mechanical axis of the femur or the step of storing in the memory the mechanical axis of tibia. The method may include the step of obtaining images of body parts, the step of registering items, or the steps of locating and registering body structure. Finally, the method may include the step of mounting a fiducial to a body part or the step of displaying the constructed plane on a monitor.

The invention further includes a process for conducting knee surgery using a surgical navigation system. The process may include one or more of the following steps, which are provided in no particular order. A first step of the method is to identify a first axis of a first bone. A second step is to track an orientation of the first axis relative to the first bone. A third step is to identify a second axis of a second bone. A fourth step is to track an orientation of the second axis relative to the second bone. A fifth step is to place the second bone in about 90 degrees of flexion relative to the first bone. A sixth step is to construct a plane through the second axis and orthogonal to the first axis. A seventh step is to track an orientation of the constructed plane. An eighth step is to expose bones in a vicinity of a knee joint. A ninth step is to measure an angular rotation of an item relative to the constructed plane and the second axis. Items may include, but are not limited to, tools, instruments, trial components, and prosthetic devices. A tenth step is to at least partially resect the first bone. An eleventh step is to close the exposed knee. An optional step may be to attach a surgical implant to the at least partially resected first bone.

In view of the foregoing, it will be seen that the several advantages of the invention are achieved and attained.

The embodiments were chosen and described in order to best explain the principles of the invention and its practical application to thereby enable others skilled in the art to best utilize the invention in various embodiments and with various modifications as are suited to the particular use contemplated.

As various modifications could be made in the constructions and methods herein described and illustrated without departing from the scope of the invention, it is intended that all matter contained in the foregoing description or shown in the accompanying drawings shall be interpreted as illustrative rather than limiting. For example, while some embodiments are illustrated in conjunction with total knee arthroplasty (TKA), those of ordinary skill in the art would understand that the invention may equally be applied to unicompartmental knee arthroplasty (UKA), bicompartmental knee arthroplasty, or articulating joint resurfacing. Thus, the breadth and scope of the present invention should not be limited by any of the above-described exemplary embodiments, but should be defined only in accordance with the following claims appended hereto and their equivalents.

1. A system for performing computer assisted surgery, the system comprising:
a. a first fiducial operatively connected to a first part;
b. a second fiducial operatively connected to a second part;
c. at least one position and orientation sensor adapted to track said first fiducial and said second fiducial;
d. a computer having a memory, a processor, and an input/output device, said input/output device adapted to receive data from said at least one position and orientation sensor relating to a position and an orientation of said first fiducial and said second fiducial, said processor adapted to process said data to identify a first axis of the first part and a second axis of the second part, and said processor adapted to construct a reference plane through said second axis and orthogonal to said first axis; and
e. a monitor operatively connected to said input/output device of said computer, and wherein said monitor is adapted to display a rendering of said reference plane.

2. The system for performing computer assisted surgery according to claim 1, further comprising an item and a third fiducial operatively connected to said item, and wherein said at least one position and orientation sensor is adapted to track said third fiducial, said input/output device is adapted to receive data from said at least one position and orientation sensor relating to a position and an orientation of said third fiducial, and said processor is adapted to calculate an angular rotation of said item relative to said reference plane.

3. The system for performing computer assisted surgery according to claim 2, wherein said item is selected from the group consisting of tools, instruments, trial components, and prosthetic devices.

4. The system for performing computer assisted surgery according to claim 1, wherein said computer is networked.

5. The system for performing computer assisted surgery according to claim 1, further comprising a foot pedal operatively connected to said computer.

6. The system for performing computer assisted surgery according to claim 1, wherein said monitor is a touchscreen.

7. The system for performing computer assisted surgery according to claim 1, further comprising a probe and a fourth fiducial operatively connected to said probe.

8. The system for performing computer assisted surgery according to claim 1, further comprising an imaging device.

9. The system for performing computer assisted surgery according to claim 1, wherein said at least one position and orientation sensor is an infrared sensor.

10. The system for performing computer assisted surgery according to claim 1, wherein said first fiducial and said second fiducial each include reflective elements.

11. The system for performing computer assisted surgery according to claim 1, wherein said first fiducial and said second fiducial each include active elements.

12. A computerized method for determining tibial rotation within a coordinate system, the method comprising the steps of:
a. providing a computer having a processor, a memory, and an input/output device;
b. identifying a mechanical axis of a femur;
c. identifying a mechanical axis of a tibia;
d. placing the tibia in about 90 degrees of flexion relative to the femur;
e. constructing a plane through the mechanical axis of the tibia and orthogonal to the mechanical axis of the femur;
f. identifying an orientation of the plane relative to the coordinate system;
g. storing the orientation of the plane in the memory of the computer; and
h. measuring an angular rotation of an item relative to the plane and the mechanical axis of the tibia.

13. The method according to claim 12, further including the step of storing in the memory the mechanical axis of the femur.

14. The method according to claim 12, further including the step of storing in the memory the mechanical axis of tibia.

15. The method according to claim 12, further including the step of obtaining images of body parts.

16. The method according to claim 12, further including the step of registering items.

17. The method according to claim 12, further including the steps of locating and registering body structure.

18. The method according to claim 12, further including the step of mounting a fiducial to a body part.

19. The method according to claim 12, further including the step of displaying the plane on a monitor.

20. The method according to claim 12, wherein the step of identifying a mechanical axis of a femur includes the step of locating data points corresponding to structure of the femur.

21. The method according to claim 12, wherein the step of identifying a mechanical axis of a tibia includes the step of locating data points corresponding to structure of the tibia.

22. A computerized method for determining tibial rotation within a coordinate system, the method comprising the steps of:
a. providing a computer having a processor, a memory, and an input/output device;
b. mounting a first fiducial to a femur;
c. identifying a mechanical axis of the femur, which includes the step of locating data points corresponding to structure of the femur;
d. mounting a second fiducial to a tibia;
e. identifying a mechanical axis of the tibia, which includes the step of locating data points corresponding to structure of the tibia;
f. placing the tibia in about 90 degrees of flexion relative to the femur;
g. sensing a position for each of the first fiducial and the second fiducial;
h. constructing a plane through the mechanical axis of the tibia and orthogonal to the mechanical axis of the femur;
i. identifying an orientation of the plane relative to the coordinate system;
j. storing the orientation of the plane in the memory of the computer; and
k. measuring an angular rotation of an item relative to the plane and the mechanical axis of the tibia, wherein said item is selected from the group consisting of tools, instruments, trial components, and prosthetic devices.
